(19) United States
(12) Patent Application Publication
(10) Pub. No.: US 2024/0287619 A1
(43) Pub. Date: Aug. 29, 2024

(54) TEMPERATURE RESPONSIVE TAGS FOR FOOD ITEMS

(71) Applicant: MICROSOFT TECHNOLOGY LICENSING, LLC, Redmond, WA (US)

(72) Inventors: Yuan-Jyue CHEN, Seattle, WA (US); Jake Allen SMITH, Seattle, WA (US); Bichlien Hoang NGUYEN, Seattle, WA (US)

(21) Appl. No.: 18/114,198

(22) Filed: Feb. 24, 2023

(51) Int. Cl. C12Q 1/6888; C12Q 1/6834; G01N 21/64

(52) U.S. Cl. CPC C12Q 1/6888; C12Q 1/6834; G01N 21/6428; G01N 2021/6439

(57) ABSTRACT

The melting temperature of double-stranded polynucleotides is used to create temperature-sensitive tags for tracking the exposure of food items to high temperatures. Once a food item is exposed to a temperature that exceeds the melting temperature of the double-stranded polynucleotides, the two strands melt and then reanneal in a different conformation. This change in conformation can be detected by fluorophores attached to one of the polynucleotide strands. The double-stranded polynucleotides may be applied directly to food items as an edible tag. Alternatively, a tag that includes a substrate with bound polynucleotides may be attached like a sticker to the food items. This provides an economical and small device for determining if individual food items were exposed to temperatures that could negatively affect quality or safety.

### BACKGROUND

Many food items can be damaged by excessive heat. Storing or transporting food at elevated temperatures may reduce shelf-life and harm food quality. Distributors, retailers, and consumers wish to know if a food item experienced higher than ideal temperatures.

Thermometers measure current temperature but do not provide a record of past temperatures. Electronic systems for measuring and recording past temperature typically include a thermometer, some type of memory, a display, and a power source. These systems may be suitable for high-value items such as vaccines or rare wines but are not economically practical for most food items. Also, the size and bulk of electronic systems generally prevent application to individual food items.

Labels are applied to individual food items either as packaging that contains the food item or as a sticker placed on the surface of the food item. These labels are typically static barcodes. A static barcode is not able to record events that happen after the label has been generated. Thus, the current types of labels used on food items cannot create a record of past temperatures.

Accordingly, at present there are no effective tags for determining if a food item was exposed to a potentially damaging high temperature. This disclosure is made with respect to these and other considerations.

### SUMMARY

This disclosure provides temperature responsive tags for food items that use the melting of double-stranded polynucleotides such as deoxyribonucleic acid (DNA) to respond to and record elevated temperatures. The polynucleotide tags are designed to have specific, predetermined melting temperatures. When exposed to a temperature higher than the melting temperature, the double-stranded polynucleotide separates into two single-stranded molecules. Once the temperature decreases, the single-stranded molecules reassemble into different structures. This change in the structure of polynucleotides provides a record of temperature events. Techniques such as fluorescent reporters and Polymerase Chain Reaction (PCR) amplification are used to detect the changed structures.

One technique uses polynucleotide tags that can be applied directly to the food item. The polynucleotides are at least partially double stranded and this double-stranded portion melts at a specific predetermined temperature. To protect the polynucleotide tags they may be encapsulated in microcapsules. Both the polynucleotide tags and any encapsulation including the microcapsules may be edible creating a type of tag that does not need to be removed from a food item before consumption.

If the food item is exposed to a temperature that melts the double-stranded portion, the polynucleotide strands reanneal into a different conformation. This different conformation can include, or through additional processing is made to include, a single-stranded polynucleotide that is referred to as an invading strand. The invading strand is a single-stranded polynucleotide that was not present before exposure to the threshold temperature.

The polynucleotide strands in the changed conformation that includes the invading strand are collected from the food item. The polynucleotide strands may then be isolated and cleaned which can include removal from encapsulation. The polynucleotide tag is then applied to a double-stranded polynucleotide structure that is attached to a substrate such as a type of paper that can bind DNA.

These double-stranded polynucleotide structures have one strand that is attached to the substrate and one strand that is attached to a fluorophore. The fluorophore gives off visible light when excited with light of a specific wavelength such as ultraviolet. Initially all the fluorophores are anchored to the substrate at a specific spot which emits visible fluorescence when illuminated. The invading strand hybridizes to an overhang region, like a “tail,” which is a single-stranded portion of one strand of the double-stranded polynucleotide structure. Because the invading strand hybridizes better than the two strands of the double-stranded polynucleotide structure, it causes strand displacement which separates the two strands of the double-stranded polynucleotide structure. The displaced strand includes the fluorophore which is now no longer attached to the substrate.

The substrate may be washed to remove the fluorophores. Without the fluorophores, there is a loss of fluorescence at the spot where they were previously bound to the substrate. Thus, the loss of fluorescence indicates that the food item experienced a temperature at least as high as the threshold temperature. The food item may be tagged with multiple different polynucleotide tags that melt at different temperatures. And the substrate may have multiple sets of double-stranded structures attached at different spots that each interacts with a different one of the polynucleotide tags. With this conformation, the pattern of which spots lose fluorescence indicates which of multiple threshold temperatures were reached (e.g., 30° C., 40° C., and 50° C.).

Another technique attaches both the polynucleotide tags and a substrate to the food item or packaging. With this technique, it is possible to determine what threshold temperature the food item was exposed to without collecting and processing the polynucleotide tags. However, because the substrate is included this implementation the tag is not edible.

The polynucleotide tags are double stranded with a fluorophore strand attached to a fluorophore and a quencher strand attached to a quencher. The quencher prevents the fluorophore from fluorescing. When the two strands of the polynucleotide tags are hybridized to each other, the quencher is positioned close to the fluorophore and there is no fluorescence. The nucleotide sequences of these polynucleotide tags are designed to melt at the threshold temperature which separates the fluorophore strand from the quencher strand.

The substrate has single-stranded polynucleotides bound to it that will hybridize to the fluorophore strands. If the food item experiences a temperature above the threshold temperature, some of the fluorophore strands will hybridize to the substrate-bound polynucleotides. This creates localized fluorescence at a spot on the substrate where single-stranded polynucleotides are bound. Thus, localized fluorescence indicates that the food item was exposed to the predetermined melting temperature. This implementation may also include a substrate with different spots that each interacts with a different polynucleotide tag. The location of fluorescence then indicates which threshold temperatures were reached.

This Summary is provided to introduce a selection of concepts in a simplified form that are further described below in the Detailed Description. This Summary is not intended to identify key features or essential features of the claimed subject matter nor is it intended to be used to limit the scope of the claimed subject matter. The term “techniques,” for instance, may refer to system(s) and/or method(s) as permitted by the context described above and throughout the document.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items. The figures are schematic representations and items shown in the figures are not necessarily to scale.

FIG. 1 illustrates application of a polynucleotide tag to a food item and use of the polynucleotide tag to record if the food item experienced a temperature that exceeds a temperature threshold.

FIG. 2 illustrates a substrate with multiple locations that are each associated with a threshold temperature and that can be used with the polynucleotide tags of this disclosure.

FIG. 3 illustrates an invading strand displacing a polynucleotide strand attached to a fluorophore from a substrate.

FIG. 4 illustrates an implementation of a polynucleotide tag in which a toehold blocker strand moves from a first polynucleotide strand to a second polynucleotide strand upon exceeding a threshold temperature.

FIGS. 5A and 5B illustrate an implementation of a polynucleotide tag in which blocker strands from two polynucleotide strands hybridize to each other and the two polynucleotide strands also hybridize to each other along part of their length upon exceeding a threshold temperature.

FIG. 6 illustrates a label that has a single-stranded polynucleotide attached to a substrate and a fluid chamber that holds a double-stranded polynucleotide with one strand attached to a fluorophore and one strand attached to a quencher.

FIG. 7 illustrates an implementation of a label in which polynucleotide tags that melt at different temperatures are mixed together in a fluid chamber.

FIG. 8 illustrates an implementation of a label in which there are multiple fluid chambers each containing a single type of polynucleotide tag.

FIG. 9 is a flow diagram showing an illustrative process for using a polynucleotide tag to determine if a food item was exposed to temperatures that exceeded a threshold temperature.

### DETAILED DESCRIPTION

The Food and Agriculture Organization (FAO) of the United Nations indicates that about 40% of food grown today is simply wasted. One reason for food waste is exposure of food items to unexpected conditions such as higher-than-ideal temperatures during storage and/or transport. Higher-than-ideal temperatures may cause spoilage or damage and harm consumer confidence in food quality. It would be best to ensure that food items are not exposed to overly high temperatures between farm and table. However, if there are unexpected conditions, the next best thing is to have a record of any high-temperature exposure.

If the exposure of a food item to temperatures higher than desired can be readily identified, mitigation efforts may be used to reduce waste. For example, food intended for human consumption may be used instead as animal feed. Alternatively, food that might have been served fresh may be used instead in a processed food product or cooked food product. Knowledge a food item's temperature history will allow distributors, retailers, chefs, and consumers to make the best use of a food item and minimize waste.

Conversely, knowing that a food item was not exposed to excessive temperatures is also useful. The assurance that a food item with an unusual color or shape was stored at suitable temperatures may give chefs and consumers the confidence to use that food item-saving it from the compost bin.

The solution provided in this disclosure for tracking temperature exposure of food items is “temperature-responsive DNA tags” that can record temperature events. DNA tags are carefully designed to have pre-determined melting temperatures. If they are exposed to temperatures higher than melting temperatures, the structures of the original DNA tags will be melted, and they reassemble into another structure. The temperature record encoded by these DNA tags can be read back conveniently using visual indications generated by fluorophores. This results in temperature-sensitive tags that are much simpler and cheaper compared to other molecule-based technology, which typically requires expensive instruments for reading.

In many implementations, the polynucleotide tags provided in this disclosure will be made of DNA, but they are not limited to DNA. Polynucleotide tags may be made out of other types of polynucleotides that are capable of forming double-stranded structures through complementary base pairing. Accordingly, polynucleotides, as used herein, include both DNA, ribonucleic acid (RNA), and hybrids containing mixtures of DNA and RNA. DNA and RNA include nucleotides with one of the four natural bases cytosine (C), guanine (G), adenine (A), thymine (T), or uracil (U) as well as unnatural bases, noncanonical bases, and modified bases. Polynucleotides also include molecules made in whole or part from nucleic acid analogs that have different structures (e.g., different backbones) than natural polynucleotides such as peptide nucleic acids (PNA), glycol nucleic acid (GNA), threose nucleic acid (TNA).

Detail of procedures and techniques not explicitly described or other processes disclosed of this application are understood to be performed using conventional molecular biology techniques and knowledge readily available to one of ordinary skill in the art. Specific procedures and techniques may be found in reference manuals such as, for example, Michael R. Green & Joseph Sambrook, Molecular Cloning: A Laboratory Manual, Cold Spring Harbor Laboratory Press, 4^{th} ed. (2012).

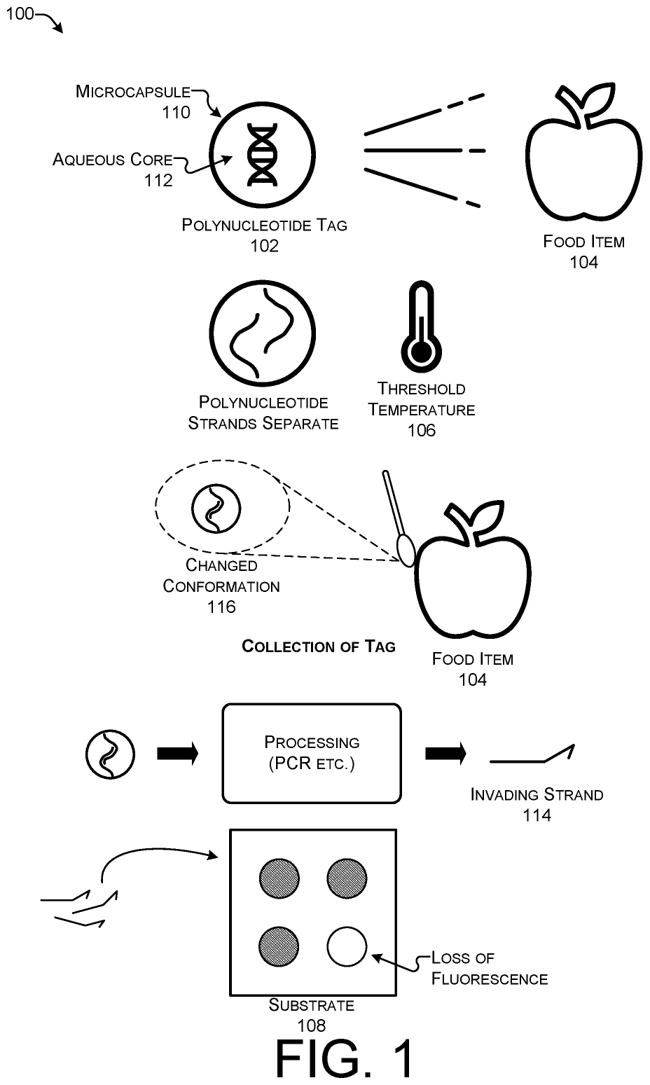

FIG. 1 is a diagram 100 that shows application of a polynucleotide tag 102 to a food item 104, change in conformation 116 of the polynucleotide tag 102 following exposure to a threshold temperature 106 to detect exposure to a temperature at least as high as the threshold temperature 106. In some implementations, the polynucleotide tag 102 is protected by encapsulation in a microcapsule 110 that contains an aqueous core 112. The food item 104 can be any type of food such as a solid item like an apple or carrot, a powdered or granular item like sugar or flour, or a liquid such as milk or wine.

The polynucleotide tags 102 themselves are also edible. That is, the polynucleotide tags 102, and any encapsulation or stabilizing agents, are safe for human consumption. Examples of edible molecules include types of molecules that are normally present in foods such as DNA, RNA, proteins, and sugars. The polynucleotide tags 102 may be designed so that the nucleotide sequences are non-natural and have no biological activity. Additionally, the total quantity of all polynucleotide tags 102 added to the food item 104 may be very small relative to size of the food item 104 itself (e.g., less than 1 g, less than 1 mg, less than 1 μg, or less than 1 ng) so that any biological activity would result in no physiological effect in a person who consumed the polynucleotide tag 102.

The polynucleotide tag 102 is at least partially double stranded and may be a fully double-stranded molecule. The double-stranded portion of the polynucleotide tag 102 is designed to have a specific melting temperature. The melting temperature, or Tₘ, is the predicted temperature at which 50% of the polynucleotides are paired with their complementary sequence. Techniques for designing polynucleotides with specific melting temperatures are well known to those of ordinary skill in the art. See M. R. Bakhtiarizadeh, M. J. Najaf-Panah, H. Mousapour, S. A. Salami, Versatility of different melting temperature (Tₘ) calculator software for robust PCR and real-time PCR oligonucleotide design: a practical guide Gene Rep, 2 (2016), pp. 1-3, 10.1016/j.genrep.2015.11.001; S. Chavali, A. Mahajan, R. Tabassum, S. Maiti, D. Bharadwaj Oligonucleotide properties determination and primer designing: a critical examination of predictions, Bioinformatics, 21 (20) (2005), pp. 3918-3925, 10.1093/bioinformatics/bti633; SantaLucia, A unified view of polymer, dumbbell, and oligonucleotide DNA nearest-neighbor thermodynamics, Proc Natl Acad Sci, 95 (4) (1998), pp. 1460-1465, 10.1073/pnas.95.4.1460; and J. SantaLucia Jr., D. Hicks, The thermodynamics of DNA structural motifs, Annu Rev Biophys Biomol Struct, 33 (2004), pp. 415-440, 10.1146/annurev.biophys.32.110601.141800.

The polynucleotides in the polynucleotide tag 102 may have any length. Typically, they will be at least about 10 nucleotides long. The polynucleotides may be created by any known or later developed technique for synthesizing polynucleotides. Methods for polynucleotide synthesis include solid-phase phosphoramidite synthesis, microchip-based oligonucleotide synthesis, ligation-mediated assembly, polymerase chain reaction (PCR)-mediated assembly, and the like. Examples of polynucleotide synthesizers 106 include ABI 394 DNA Synthesizer (Applied Biosystems, Foster City, Calif.), the Piezoelectric Oligonucleotide Synthesizer and Microarrayer (POSAM), photolithographic oligoarray synthesizers, etc. Polynucleotides may also be synthesized by enzymatic processes. The enzyme terminal deoxynucleotidyl transferase (TDT) used in enzymatic polynucleotide synthesis is known to generate random sequences when provided with ar mixture of nucleoside bases. See Fowler J D, Suo Z (2006) Biochemical, Structural, and Physiological Characterization of Terminal Deoxynucleotidyl Transferase. Chemical Reviews 106(6):2092-2110.

Currently with phosphoramidite synthesis, the length of the polynucleotides will be less than about 300 nucleotides. Thus, the length of the polynucleotide tags 102 may be about 10-300 nucleotides, such as about 100 nucleotides or about 200 nucleotides. The polynucleotide tags 102 may be created by enzymatic synthesis which can create longer strands up to about 10,000 nucleotides. Thus, the length of the polynucleotide tags 102 may be about 10-10,000 nucleotides, such as about 300 nucleotides, about 400 nucleotides, or about 1000 nucleotides.

The food item 104 may be labeled with a large number of copies of the same polynucleotide tag 102. For example, the food item 104 may be labeled with many thousands, tens of thousands, hundreds of thousands, millions, or billions of synthetic polynucleotides. The original synthesis may create many copies of the polynucleotide tag 102 with the same sequence. Additionally or alternatively, the one or more copies of the polynucleotide tag 102 may be duplicated to further increase the number of molecules with that same sequence. Any one of multiple enzymatic techniques known to those of ordinary skill in the art may be used to increase the number of copies of a polynucleotide. For example, the well-known technique of PCR may be used to exponentially amplify polynucleotides. Isothermal amplification methods may also be used. Isothermal methods typically employ unique DNA polymerases for separating duplex DNA. Isothermal amplification methods include Loop-Mediated Isothermal Amplification (LAMP), Whole Genome Amplification (WGA), Strand Displacement Amplification (SDA), Helicase-Dependent Amplification (HDA), Recombinase Polymerase Amplification (RPA), and Nucleic Acid Sequences Based Amplification (NASBA). See Yongxi Zhao, et al., Isothermal Amplification of Nucleic Acids, Chemical Reviews, 115 (22), 12491-12545 (2105) for a discussion of isothermal amplification techniques.

The polynucleotide tag 102 may be placed directly onto the food item 104 or mixed in the food item 104 if the food item 104 is a powder or liquid. Cultured or synthetic foods may have the polynucleotide tags 102 added as part of a standard manufacturing process. Because of the small size, polynucleotide tags 102 can be easily sprayed onto a food item 104. The food item 104 may also be dipped in a liquid containing many copies of the polynucleotide tag 102. The liquid may be water or an aqueous solution such as a buffer solution. The processing of many food items already includes a step of spraying or coating the food item 104 with a liquid such as a solution of chemicals to promote or prevent ripening. Polynucleotide tags may also be added to food items 104 for other purposes such as inventory tracking as described in U.S. patent application Ser. No. 17/511,454 filed on Oct. 26, 2021. Thus, the polynucleotide tag 102 may be mixed with a liquid that will be applied to the food item 104 as a part of processing that would occur even if a temperature-sensitive tag was not added.

Alternatively, the polynucleotide tag 102 may be placed on packaging that contains the food item 104. In some implementations described later, the polynucleotide tags 102 are combined with the substrate 108 into a device that may be similar to a sticker or label. These devices are not edible and cannot be directly applied to some food items 104 such as liquids, powders, meat, and fish.

The polynucleotide tag 102 may be applied “naked” without any protection or modification. Thus, in one implementation, naked double-stranded (including partially double-stranded) DNA is placed on the food item 104.

Alternatively, polynucleotide tags 102 may be protected with stabilizing agents or encapsulated by a protective coating such as within a microcapsule 110. One example of a stabilizing agent is trehalose which is known to have a strong stabilizing effect on DNA secondary structure. See Bonnet J, Colotte M, Coudy D, Couallier V, Portier J, Morin B, Tuffet S. Chain and conformation stability of solid-state DNA: implications for room temperature storage. Nucleic Acids Res. 2010 March; 38(5):1531-46. Protection enables the polynucleotide tags 102 to remain undamaged for longer than unprotected polynucleotides.

Encapsulation such as in a water-in-oil emulsion is another way to protect the polynucleotide tags 102. Encapsulation may include formation of microcapsules 110 around aqueous solution that contains the polynucleotide tags 102. Microcapsules 110 are capsules that have a diameter measured in micrometers which is typically less than about 2000 micrometers. Nanocapsules are capsules that have a diameter measured in nanometers which is typically less than about 500 nanometers. As used herein, microcapsules 110 include nanocapsules. The microcapsules 110 include an aqueous core 112 that can contain a buffer solution or other fluid suitable for storing polynucleotides. The aqueous environment allows the polynucleotide tag 102 to respond to kinetic energy provided by elevated temperatures and disassociate, or melt, into two separate strands when the threshold temperature 106 is reached. Techniques for encapsulating polynucleotides in microcapsules are known to those of ordinary skill in the art. One example of a suitable technique is provided in Lambert, G., Fattal, E., Pinto-Alphandary, H. et al., Polyisobutylcyanoacrylate Nanocapsules Containing an Aqueous Core as a Novel Colloidal Carrier for the Delivery of Oligonucleotides. Pharm Res 17, 707-714 (2000).

Multiple techniques for stably storing polynucleotides other than encapsulation are also known to those of ordinary skill in the art. For example, polynucleotides may be mixed with salts or saccharides to improve stability. Any suitable technique may be adapted for use with the food item 104 depending on the composition of the food item 104.

The double-stranded portion of the polynucleotide tags 102 is designed with a melting temperature that is the threshold temperature 106. Thus, once the threshold temperature 106 is reached or exceeded the double-stranded polynucleotide tag 102 separates into two single-stranded molecules. Some portion of the polynucleotide tags 102 will melt at temperatures slightly below the threshold temperature 106; however, this generally will not affect detection or lead to false positive results. It may lead to a weak signal that can be interpreted as the food item 104 experiencing a temperature close to but not exceeding the threshold temperature 106.

The threshold temperature 106 may be any temperature at which double-stranded polynucleotides dissociate into separate single-stranded molecules. For example, the threshold temperature 106 may be any temperature between about 25-95° C. Techniques for designing a double-stranded polynucleotide with a specific Tₘ are known to those in the art and include adjusting G-C content and the length of double-stranded hybridization.

Generally, the temperatures experienced by the food item 104 will be lower than the threshold temperature 106. Thus, if there is an unexpected event that results in the food item 104 being exposed to high temperatures, the temperature will again drop below the threshold temperature 106. Upon cooling if the two separate strands reannealed in the same conformation there would be no record of the exposure to an elevated temperature. Accordingly, the polynucleotide tags 102 are designed so that at least some of the polynucleotide strands do not reanneal but take a changed conformation 116. The changed conformation 116 may be created by having multiple different polynucleotide strands as the polynucleotide tag 102 such that different combinations anneal to each other after heating and cooling. Example implementations are described in detail later in this disclosure. It is also not required for all of the polynucleotide tags 102 to have a changed conformation 116 so long as the quantity that does is sufficient to cause a detectible change in fluorescence.

The polynucleotide tag 102 is collected from the food item 104 at some point after storage and/or transport. For example, the polynucleotide tag 102 may be collected after the food item 104 is delivered to a grocery store or restaurant. The specific technique for collecting the polynucleotide tag 102 will depend on how the polynucleotide tag 102 is applied to the food item 104. For example, the polynucleotide tag 102 may be collected from the food item 104 by swabbing the surface, removing a portion of the food item 104 (e.g., if the food is a liquid or powder) and extracting the polynucleotide tag 102, rinsing the food item 104 and extracting the polynucleotide tag 102 from the rinse solution, or by any other suitable technique. Many techniques and commercial kits for collecting and purifying DNA and other polynucleotides are known to those of ordinary skill in the art. For example, techniques developed for environmental or forensic samples of polynucleotides may be used to collect and process the polynucleotide tag 102 collected from the food item 104. See Hinlo R., Gleeson D., Lintermans M., Furlan E. (2017) Methods to maximise recovery of environmental DNA from water samples. PLoS ONE 12(6) and Butler, John M. Forensic DNA Typing—Biology, Technology, and Genetics of STR Markers” Second Edition, Elsevier Academic Press, Burlington, MA (2005).

The polynucleotide tag 102 in the changed conformation 116 collected from the food item either includes an invading strand 114 or is processed to create an invading strand 114. The processing may include PCR and/or other well-known and conventional techniques for manipulating polynucleotides. However, the processing is not part of every implementation. An invading strand 114 is a single-stranded polynucleotide that through strand displacement “invades” another double-stranded polynucleotide and displaces one of those strands. Thus, the invading strand 114 swaps places with one of the strands of a double-stranded polynucleotide. The strand displacement causes a fluorophore that is attached to the surface of the substrate 108 to separate from the substrate 108. This can result in a detectable loss of fluorescence.

The loss of fluorescence is the signal that the food item 104 experienced the threshold temperature 106. The substrate 108 may have multiple locations that each interact with different polynucleotide tags 102 which melt at different temperatures. Thus, the substrate 108 can report on which of multiple threshold temperatures 106 were reached. For each threshold temperature that was reached there will be a loss of fluorescence at the corresponding location on the substrate 108. Thus, with these techniques melting and hybridization of polynucleotides are used to create a record of temperatures experienced by a food item 104 without polynucleotide sequencing or other complex analysis of the polynucleotide tags 102.

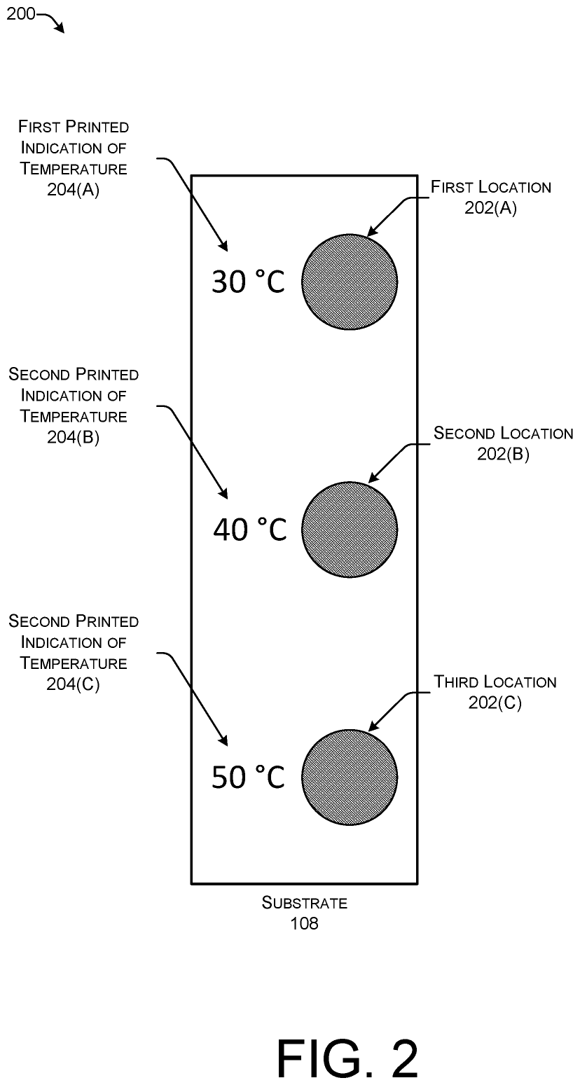

FIG. 2 is an illustration of a device 200 that exhibits localized changes in fluorescence when contacted by polynucleotide strands having specific melting temperatures. This device 200 can be implemented as a substrate 108 with substrate-bound polynucleotides having specific sequences attached at one or more discrete locations 202, or “spots.” Each spot is associated with a different threshold temperature.

This example shows a first location 202(A) associated with a first threshold temperature, a second location 202(B) associated with a second threshold temperature, and a third location 202(C) associated with a third threshold temperature. However, there may be a lesser or greater number of locations 202 including only a single location. Although shown as circles, the spots or locations 202 may be any shape.

The substrate 108 may be formed from any suitable substance to which polynucleotides can be attached. For example, the substrate 108 may be made from paper or a thin membrane. The substrate 108 may also be made from silicon or a synthetic polymer such as a plastic. In some implementations, the polynucleotides may be attached directly to the substrate 108. In other implementations, the substrate 108 may be functionalized with other molecules that attach to the polynucleotides. Multiple techniques are known to those of skill in the art for attaching polynucleotides to a substrate at specific locations. Persons of ordinary skill in the art can readily identify an appropriate technique for a given type of substrate 108.

In one implementation, the substrate 108 is a nitrocellulose or nylon membrane. Techniques adapted from Southern blotting are used to attach the substrate-bound polynucleotides to the substrate 108. Specifically, ion exchange interactions bind the polynucleotides to the membrane due to the negative charge of the polynucleotides and the positive charge of the membrane. The substrate-bound polynucleotides may be synthesized with a poly-T tail (e.g., about 30 bases long) on the 5′-ends to increase the strength of electrostatic binding with the nitrocellulose or nylon membrane.

In some implementations, the substrate 108 includes printed indications of temperature 204 adjacent or proximate to one or more of the locations 202 where substrate-bound polynucleotides are attached. These printed indications of temperature 204 may be human-readable to facilitate interpreting the meaning of fluorescence at the one or more locations 202. Specifically, the printed indication of temperature 204 can show the threshold temperature for polynucleotide tags that hybridize to substrate-bound polynucleotides at the corresponding spot. Thus, the first location 202(A) may be associated with a first printed indication of temperature 204(A) (e.g., 30° C.), the second location 202(B) may be associated with a second printed indication of temperature 204(B) (e.g., 40° C.), and the third location 202(C) may be associated with a third printed indication of temperature 204(C) (e.g., 50° C.).

Thus, the substrate 108 has multiple locations 202 that will either lose or gain fluorescence depending on which polynucleotides are brought into contact with the substrate 108. If there is more than one “spot,” then the location of the fluorescence change is indicative of which threshold temperatures were experienced.

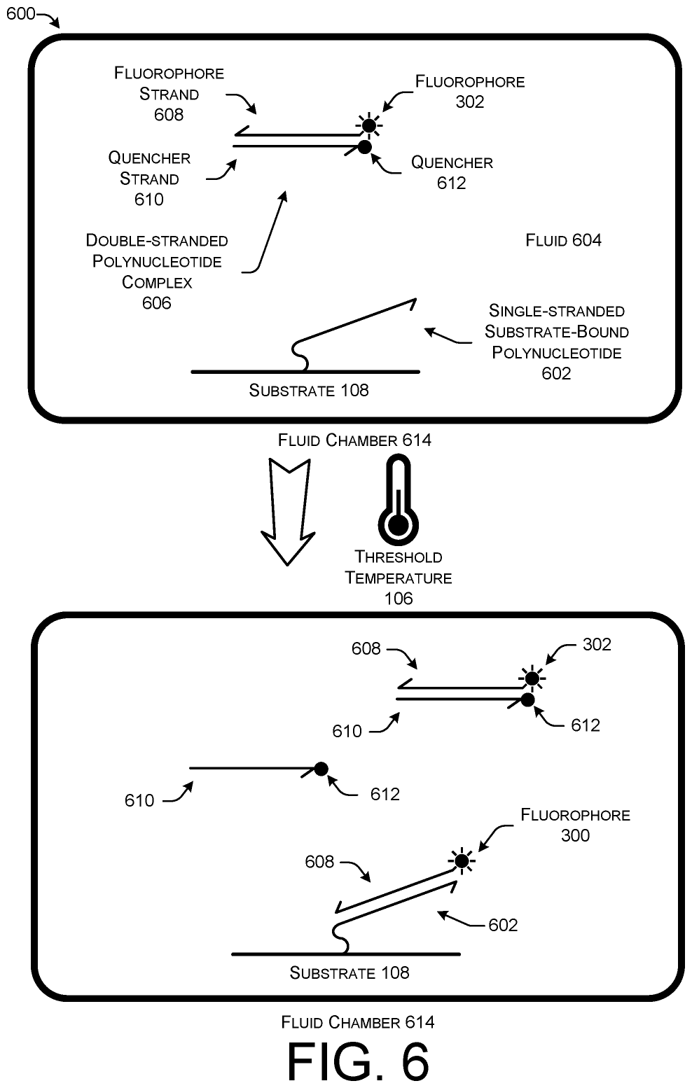

There are two ways in which the device 200 containing the substrate 108 may be used. The first way is for a post-sampling reading of polynucleotide tags collected from a food item as shown in FIG. 1. With this technique, the substrate 108 is used separately from the food item. The substrate 108 is like a testing strip that reports on what conformation of polynucleotide tags (and thus what temperatures were reached) are collected from the food item. The other way is to use the device 200 with the substrate 108 as a label applied to the food item or its packaging. This implementation combines the substrate 108, the polynucleotide tags, and fluid in contact with the substrate 108 as a single device. This integrated label-type device can exhibit changes in fluorescence without any user manipulation such as collection of the polynucleotide tags or applying collected tags to the substrate 108. Examples of this implementation are illustrated in FIGS. 6-8.

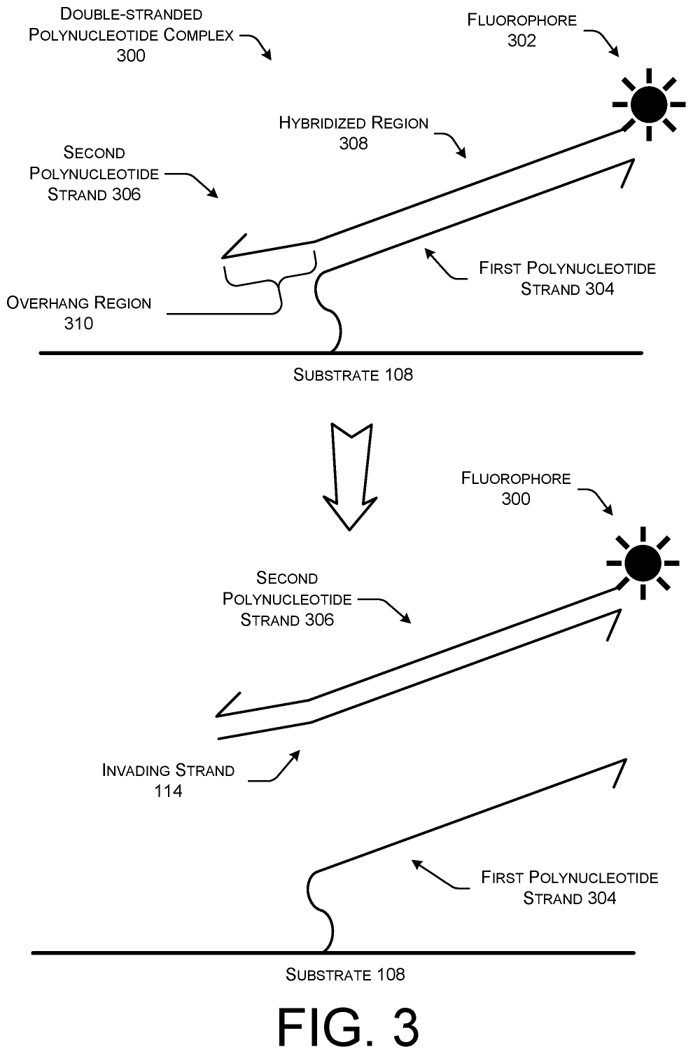

FIG. 3 shows a conformation of a substrate-bound double-stranded polynucleotide complex 300 that includes a fluorophore 302. In one implementation, these are the polynucleotides attached at discrete locations on the substrate 108 as shown in FIG. 2. The double-stranded polynucleotide complex 300 includes a first polynucleotide strand 304 that is attached to the substrate 108. This strand is also referred to as a substrate-bound polynucleotide strand. A second polynucleotide strand 306 is hybridized along a part of its length to the first polynucleotide strand 304. Thus, there is a hybridized region 308 where the two strands of the double-stranded polynucleotide complex 300 are hybridized to each other. Hybridization does not require full complementarity.

The second polynucleotide strand 306 is attached to a fluorophore 302. The fluorophore 302 may be any type of fluorophore suitable for attachment to a polynucleotide such as fluorescein, rhodamine, cyanine, and Alexa Fluor dyes. The fluorophore 302 can be attached to the second polynucleotide strand 306 through a variety of methods, such as chemical modification or incorporation during synthesis. Suitable types of fluorophores and techniques for attaching fluorophores to polynucleotides are well known to those of ordinary skill in the art.

The second polynucleotide strand 306, when part of the double-stranded polynucleotide complex 300, also includes a single-stranded region that does not hybridize to the first polynucleotide strand 304. This single-stranded region is referred to as an overhang region 310. The overhang region 310 makes toehold-mediated strand displacement possible.

Toehold-mediated strand displacement is a mechanism for controlling the hybridization of specific sequences of polynucleotides. It relies on a small, specific region of the polynucleotide strand, called a “toehold,” which allows a secondary strand to bind and displace a larger, target strand. The process works by introducing a short, single-stranded polynucleotide molecule called an invading strand 114 that is complementary to the toehold region of the target strand which is the overhang region 310. The invading strand 114 binds to the toehold, creating a stable complex between itself and the second polynucleotide strand 306.

The thermodynamics of polynucleotide hybridization plays a key role in the binding of the invading strand 114 to the toehold. The invading strand 114 binds to the overhang region 310 through complementary base pairing, which creates hydrogen bonds between the nucleotides of the invading strand 114 and the overhang region 310. The strength of these hydrogen bonds determines the stability of the complex, with stronger bonds leading to a more stable complex.

The invading strand 114 then uses the energy from the binding to the toehold to unwind the second polynucleotide strand 306 and displace it. Because the invading strand 114 has a greater number of nucleotides that hybridize to the second polynucleotide strand 306 than does the first polynucleotide strand 304, binding is stronger leading to displacement from the first polynucleotide strand 304. Displacement of the second polynucleotide strand 306 frees the fluorophore 302 from attachment to the substrate 108. The fluorophore 302 can then be washed away resulting in loss of fluorescence. Because the invading strand 114 will not exist as a single-stranded molecule unless the polynucleotide tag has taken a changed conformation following exposure to the threshold temperature, loss of fluorescence serves as an indication that the threshold temperature was reached. One example of toehold-mediated strand displacement being used for characterizing multiple message DNA strands by patterns of fluorescence is discussed in Kimberly Berk, et al., Rapid Visual Authentication Based on DNA Strand Displacement, ACS Appl. Mater. Interfaces (13) 19476-19486 (2021).

Double-stranded polynucleotide complexes 300 each with different sequences that hybridize to invading strands 114 associated with different melting temperatures may be attached to different locations 202 on the substrate as shown in FIG. 2. Thus, the example implementation of the device 200 shown in FIG. 2 can be combined with the structures of the substrate-bound polynucleotide complex 300 shown in FIG. 3. The first location 202(A) will have a first polynucleotide strand 304 attached to the substrate 108 and a second polynucleotide strand 306 hybridized to the first polynucleotide strand 304 and attached to a fluorophore 302. The second polynucleotide strand 306 also includes a first overhang region 310 that does not hybridize to the first polynucleotide strand 304.

The second polynucleotide strand 306 is configured to disassociate from the first polynucleotide strand 304 and release the fluorophore 302 from attachment to the substrate 108 upon contact with a first invading strand 114 that hybridizes to the first overhang region 310 and the remainder of the second polynucleotide strand 306. Other locations 202 on the substrate 108 will have a similar polynucleotide complex attached that hybridizes to invading strands of different sequences.

For example, the second location 202(B) will include a third polynucleotide strand (i.e., a different version of the first polynucleotide strand 304) attached to the substrate and a fourth polynucleotide strand (i.e., a different version of the second polynucleotide strand 306) hybridized to the third polynucleotide strand and attached to a fluorophore. The fourth polynucleotide strand includes a second overhang region (i.e., similar but with a different sequence than the first overhang region 310) that does not hybridize to the third polynucleotide strand. The fourth polynucleotide strand is configured to disassociate from the third polynucleotide strand and release the fluorophore from attachment to the substrate 108 upon contact with a second invading strand that hybridizes to the second overhang region and the remainder of the fourth polynucleotide strand.

The substrate-bound polynucleotides (i.e., the first polynucleotide strand 304 and the fourth polynucleotide strand, etc.) may all have the same sequence. If they do, then the hybridized region 308 of the polynucleotide strands attached to the fluorophore 302 (i.e., the second polynucleotide strand 306, and the third polynucleotide strand, etc.) will also have the same sequence. However, to make the separate locations 202 on the substrate 108 respond to different threshold temperatures there needs to be discrimination between different invading strands 114. Thus, the sequences of the overhang regions 310 must be different.

Continuing with the same nomenclature, the third location 202(C) will include a fifth polynucleotide strand attached to the substrate and a sixth polynucleotide strand hybridized to the first polynucleotide strand and attached to a fluorophore. The sixth polynucleotide strand includes a third overhang region that does not hybridize to the fifth polynucleotide strand. The sixth polynucleotide strand is configured to disassociate from the fifth polynucleotide strand and release the fluorophore from attachment to the substrate 108 upon contact with a third invading strand that hybridizes to the third overhang region and the remainder of the sixth polynucleotide strand. Any additional locations 202 or spots on a substrate 108 will have similar polynucleotides attached.

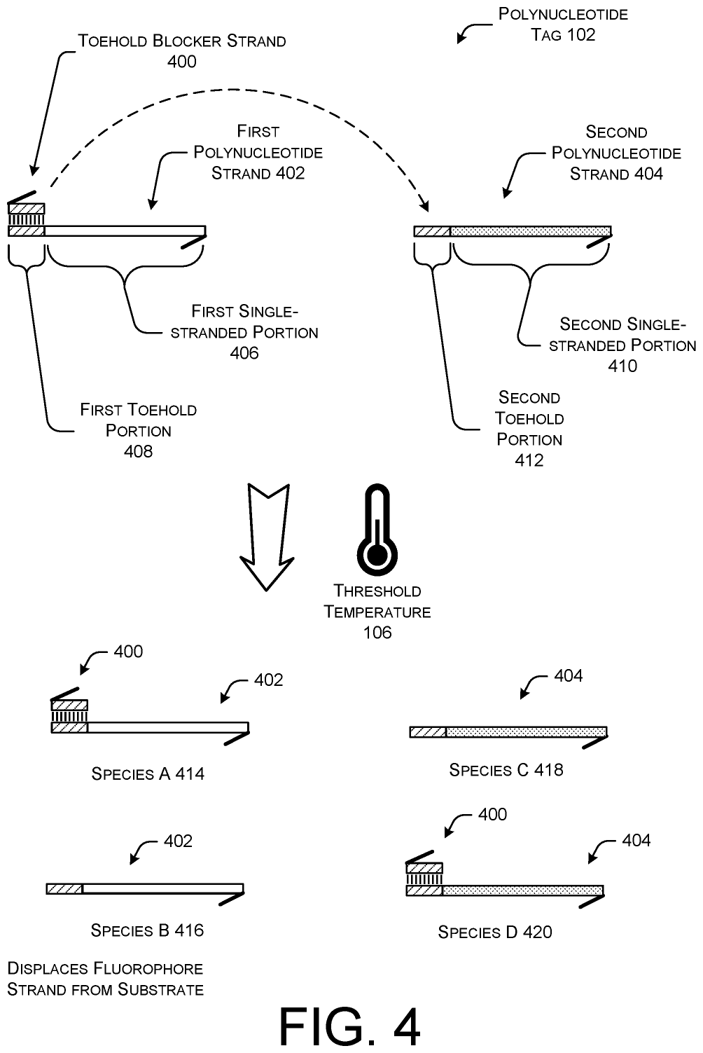

FIG. 4 shows an implementation of the polynucleotide tag 102 in which a toehold blocker strand 400 moves from a first polynucleotide strand 402 to a second polynucleotide strand 404 when heated above the threshold temperature 106. This implementation may be used with the substrate-bound double-stranded polynucleotide complexes shown in FIG. 3 and the substrate shown in FIG. 2.

In this implementation, the polynucleotide tag 102 comprises two partially doubled-stranded polynucleotides. The first polynucleotide strand 402 has a first single-stranded portion 406 and a first toehold portion 408. The first single-stranded portion 406 is generally longer than the first toehold portion 408 but it is not required to be longer. The toehold blocker strand 400 is hybridized to the first toehold portion 408. The second polynucleotide strand 404 is present initially as a single-stranded molecule. It includes a second single-stranded portion 410 and a second toehold portion 412. The second toehold portion 412 is also capable of hybridization to the toehold blocker strand 400. The second toehold portion 412 may have the same nucleotide sequence as the first toehold portion 408 but it may also have a different sequence that also hybridizes to the toehold blocker strand 400.

The Tₘ of the toehold blocker strand 400 and the first toehold portion 408 is the threshold temperature 106. If the polynucleotide tag 102 is not heated to the threshold temperature 106, then the toehold blocker strand 400 remains attached to only the first polynucleotide strand 402. If, however, the threshold temperature 106 is reached, the toehold blocker strand 400 disassociates from the first polynucleotide strand 402 and becomes free in solution. Upon cooling, the toehold blocker strand 400 may anneal to either the first polynucleotide strand 402 or the second polynucleotide strand 404. Likely, some copies of the toehold blocker strand 400 will anneal to each.

This can create four different species of polynucleotides following heating. This collection of multiple different species of double-stranded and single-stranded polynucleotides illustrated at the bottom of FIG. 4 represents the changed conformation. Species A 414 is the first polynucleotide strand 402 with the toehold blocker strand 400 reannealed. Species B 416 is the first polynucleotide strand 402 without the toehold blocker strand 400. This is a single-stranded molecule that functions as the invading strand 114 and displaces the fluorophore strand from the substrate as shown in FIG. 3. Species B 416 did not exist before heating because all copies of the first polynucleotide strand 402 were hybridized to the toehold blocker strand 400. Even though there may be some disassociation of the first polynucleotide strand 402 and the toehold blocker strand 400 below the threshold temperature 106, the amount will generally be small enough that there is no observable change in fluorescence.

There is also species C 418 that is the second polynucleotide strand 404 without the toehold blocker strand 400. There may be many copies of species C 418 if the second polynucleotide strand 404 is present in excess. This strand does not hybridize to the substrate-bound polynucleotides and does not act as an invading strand. Species D 420 may be thought of as a “sink” that adsorbs the toehold blocker strand 400. Species D 420 is the second polynucleotide strand 404 hybridized to the toehold blocker strand 400. The polynucleotide tag 102 may be designed to bias hybridization of the toehold blocker strand 400 to the second polynucleotide strand 404 rather than the first polynucleotide strand 402 in order to create more of species B 416 and less of species A 414. One way of biasing the toehold blocker strand 400 to anneal to the second polynucleotide strand 404 upon cooling is to provide many more copies of the second polynucleotide strand 404 than the first polynucleotide strand 402. Another way of causing the toehold blocker strand 400 to preferentially reanneal to the second polynucleotide strand 404 rather than back to the first polynucleotide strand 402 is for the second toehold portion 412 to hybridize more tightly to the toehold blocker strand 400 than the first toehold portion 408. This may be done may making the level of complementarity between the second toehold portion 412 and the toehold blocker strand 400 higher than the level of complementarity with the first toehold portion 408. The specific conformation of the polynucleotide tag 102 may be tuned experimentally to create a sufficient number of species B 416 molecules so that there is an observable decrease in fluorescence. For example, the ratio of the first polynucleotide strand 402 to the second polynucleotide strand 404 and/or differences in the sequences of the first toehold portion 408 and the second toehold portion 412 may be adjusted.

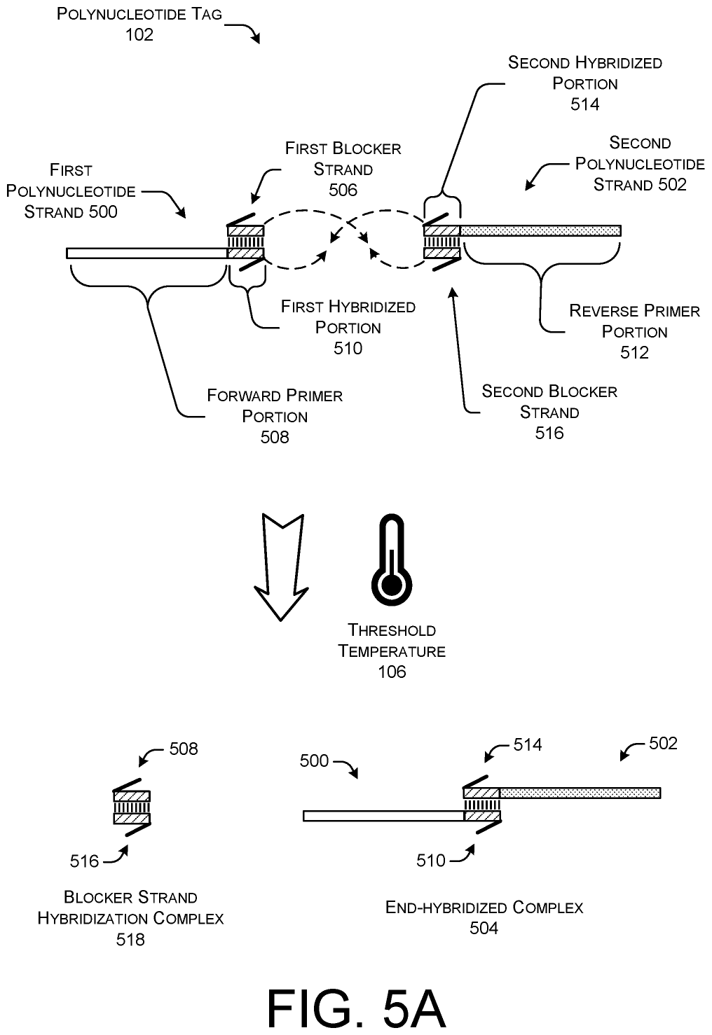

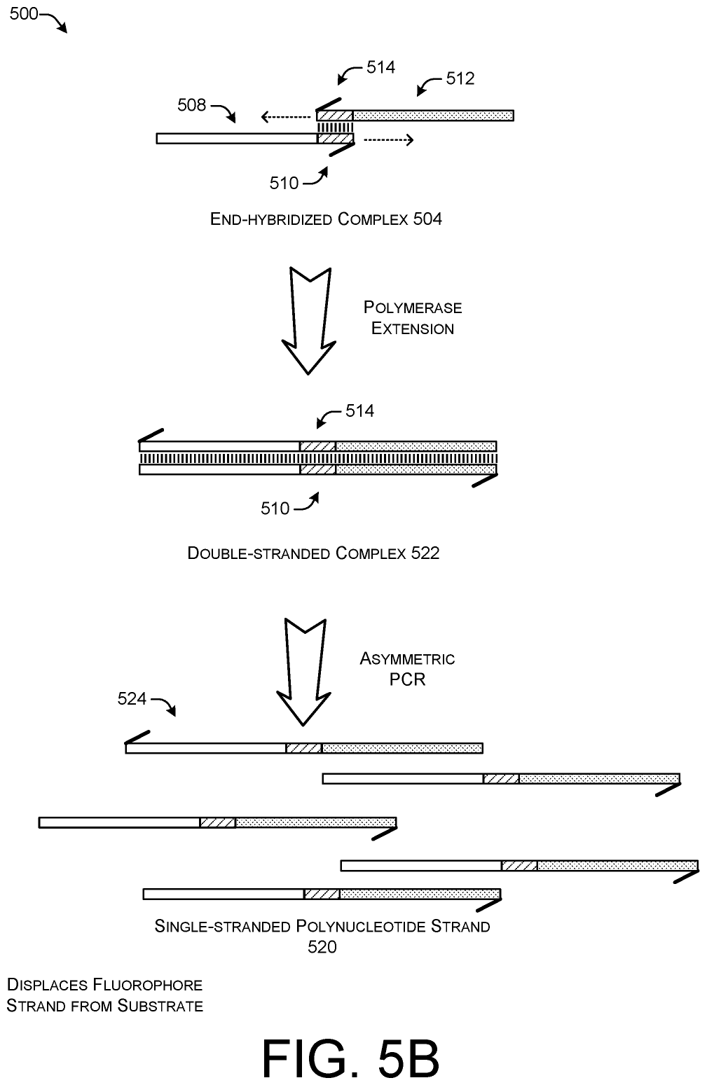

FIG. 5A shows an implementation of the polynucleotide tag 102 in which a first polynucleotide strand 500 and a second polynucleotide strand 502 hybridize to each other following heating to the threshold temperature 106 in an end-hybridized complex 504. This implementation of the polynucleotide tag 102 includes two partially double-stranded polynucleotides. The first polynucleotide strand 500 is hybridized to a first blocker strand 506. The first polynucleotide strand 500 has a forward primer portion 508 that is single stranded and a first hybridized portion 510 that is hybridized to the first blocker strand 506. The forward primer portion 508 is typically, but not necessarily, longer than the first hybridized portion 510. The second polynucleotide strand 502 has a similar structure with a reverse primer portion 512 that is single stranded and a second hybridized portion 514 that is hybridized to a second blocker strand 516.

Upon heating to the threshold temperature 106, the first blocker strand 506 and the second blocker strand 516 separate from the first polynucleotide strand 500 and the second polynucleotide strand 502 respectively. The first blocker strand 506 and the second blocker strand 516 have complementary sequences. Thus, when cooling the first blocker strand 506 and the second blocker strand 516 can anneal to each other forming a double-stranded structure. Typically, the sequences of the first blocker strand 506 and the second blocker strand 516 will be fully complementary but 100% complementarity is not required. The first hybridized portion 510 of the first polynucleotide strand 500 and the second hybridized portion 514 of the second polynucleotide strand 502 are also complementary. Thus, the ends of the first polynucleotide strand 500 and the second polynucleotide strand 502 can hybridize to each other forming the end-hybridized complex 504. The end-hybridized complex 504 is a polynucleotide structure that is not present originally and represents a changed conformation of the polynucleotide tag 102.

Hybridization over the full length of the first blocker strand 506 and the second blocker strand 516 creates an energetically favorable and stable structure. Thus, the predominant structures formed after heating and cooling are the blocker strand hybridization complex 518 and the end-hybridized complex 504. The end-hybridized complex 504 is not, however, a single-stranded molecule that can function as an invading strand.

FIG. 5B continues the implementation of FIG. 5A by showing the processing of the end-hybridized complex 504 to create a single-stranded polynucleotide strand 520 that can function as an invading strand. The double-stranded region in the middle of the end-hybridized complex 504 is extended along both strands to make a double-stranded complex 522 that is double-stranded along its full length. Any suitable technique for extending a polynucleotide by making a complementary strand to a template strand may be used. In one implementation, polymerase extension is performed on the end-hybridized complex 504. Polymerase extension uses a proofreading polymerase to accurately add single nucleotides that are complementary to the nucleotides in a template strand. In order to prevent the end-hybridized complex 504 from melting, polymerase extension is performed at a temperature less (e.g., at least 5° C. less) than the threshold temperature 106. Multiple polymerases that function at lower temperatures than standard polymerases are known to those of ordinary skill in the art including Psychromonas ingrahamii DNA polymerase I that can extend DNA at temperatures below freezing. See Xue Y, Braslavsky I, Quake SR. Temperature effect on polymerase fidelity. J Biol Chem. 2021 November; 297(5).

In one implementation, this double-stranded complex 522 may be detected by PCR amplification without the use of fluorescence. This molecule is longer than any polynucleotide present originally in the polynucleotide tag 102. PCR amplification using primers that hybridize to the forward primer portion 508 and the reverse primer portion 512 will amplify this molecule. The amplification product may be detected on a gel as a band with a size that would not be present if the polynucleotide tag 102 was not heated to the threshold temperature 106.

The double-stranded complex 522, because it is double stranded, does not function as an invading strand. Accordingly, further processing is used to create the single-stranded polynucleotide strand 520 that can displace a fluorophore strand as shown in FIG. 3. One technique is amplification of the double-stranded complex 522 by asymmetric PCR. Asymmetric PCR is a method of amplifying polynucleotide sequences where the amount of one of the primers is significantly higher than the other primer. This creates a bias in the amplification process, resulting in a larger amount of one of the polynucleotide strands compared to the other. This method provides a simple and efficient way to produce a large number of single-stranded polynucleotides. This is used to create many more copies of the single-stranded polynucleotide strand 520 than its complementary strand 524. Because there are more copies of this strand, many will not form the double-stranded complex 522 and remain single stranded so they can function as invading strands. Loss of fluorescence can result which would not occur if the threshold temperature 106 was not reached and the end-hybridized complex 504 was not formed.

FIG. 6 illustrates a device 600 that uses polynucleotides to report high-temperature exposure. This device 600 may be implemented as a label that can be affixed to a food item or packaging for a food item. The device 600 includes a substrate 108 with a single-stranded substrate-bound polynucleotide 602 attached to the substrate 108. The single-stranded substrate-bound polynucleotide 602 is a single polynucleotide and initially is not part of a polynucleotide complex. This is a different structure bound to the substrate 108 than the multi-polynucleotide complexes shown in FIG. 3 and discussed in conjunction with the implementations shown in FIGS. 4 and 5. The single-stranded substrate-bound polynucleotide 602 may be attached to the substrate 108 by any suitable technique for attaching a polynucleotide to a surface including those discussed elsewhere in this disclosure.

In this device 600 there is fluid 604 in contact with the surface of the substrate 108 and the substrate-bound polynucleotide 602. The fluid 604 is compatible with polynucleotides and conducive to polynucleotides hybridizing and melting in response to temperature changes. The fluid 604 may be an aqueous solution. The fluid 604 may be a buffered solution such as, but not limited to, Tris-EDTA buffer. The fluid 604 may have buffer concentrations, salt concentrations, and a pH level of any solution commonly used for biochemical manipulations of polynucleotides. Suitable fluids are known to those of ordinary skill in the art.

Within the fluid 604 there is a double-stranded polynucleotide complex 606 with a fluorophore strand 608 attached to a fluorophore 302 and a quencher strand 610 attached to a quencher 612. This double-stranded polynucleotide complex 606 is the polynucleotide tag. The quencher 612 prevents fluorescence of the fluorophore 302. Thus, there is no fluorescence when the fluorophore strand 608 and the quencher strand 610 are hybridized to each other. The quencher strand 610, when removed, enables the fluorophore 302 on the fluorophore strand 608 to generate a fluorescent signal. The fluorophore strand 608 and the quencher strand 610 will generally be synthesized in advance and pre-annealed to form the double-stranded polynucleotide complex 606.

The double-stranded polynucleotide complex 606 is designed with a nucleotide sequence with a melting temperature that is the threshold temperature 106. Thus, if the device 600 is heated to the threshold temperature 106, the fluorophore strand 608 will disassociate from the quencher strand 610 and they will become two separate single-stranded polynucleotides free in solution. The substrate-bound polynucleotide 602 has a sequence that hybridizes to the fluorophore strand 608. Once the temperature cools below the threshold temperature 106, some portion of the fluorophore strands 608 will hybridize to the substrate-bound polynucleotide 602 instead of to one of the quencher strands 610. This creates localized fluorescence at a location on the substrate 108 where the substrate-bound polynucleotide 602 are attached. Many copies of the single-stranded substrate-bound polynucleotide 602 may be attached at the same “spot” near to each other in a cluster or in the same area of the substrate 108. This results in a sufficient number of fluorophores 302 that generate a visibly detectable signal when excited.

Some of the fluorophore strands 608 after heating to the threshold temperature 106 and subsequent cooling may reanneal with a quencher strand 610 in solution. This does not affect the readability of the device 600 so long as a sufficient number of fluorophore strands 608 hybridize to substrate-bound polynucleotides 602. In order to increase the strength of the fluorescent signal, it may be preferable to bias hybridization of free fluorophore strands 608 to the substrate-bound polynucleotide 602 instead of to a quencher strand 610. This is done by making the hybridization of the fluorophore strand 608 to a substrate-bound polynucleotide 602 more thermodynamically favorable than hybridization to a quencher strand 610. There are multiple techniques that may be employed to make one double-stranded polynucleotide paring more likely than another.

For example, the quencher strand 610 may be designed so that it has a mismatch or less than full complementarity to the fluorophore strand 608. If the substrate-bound polynucleotide 602 is fully complementary to the fluorophore strand 608, this will drive hybridization to the substrate-bound polynucleotide 602 instead of to a quencher strand 610 because of the higher degree of complementarity. Thus, the single-stranded substrate-bound polynucleotide 602 may have a higher degree of complementarity with the fluorophore strand 608 than the quencher strand 612 has with the fluorophore strand 608.

As an additional example, the length of the complementary sequence may be longer between the fluorophore strand 608 and the substrate-bound polynucleotide 602 than with the quencher strand 610. For example, in the double-stranded polynucleotide complex 606, the fluorophore strand 608 may have a short overhang region such as, but not limited to, one or two nucleotides. Thus, the fluorophore strand 608 will not hybridize along its entire length with the quencher strand 610. However, the substrate-bound polynucleotide 602 will have a sequence that hybridizes to the entire length of the fluorophore strand 608. The increased length of the complementary sequence makes hybridization to the substrate-bound polynucleotide 602 more favorable. Thus, the single-stranded substrate-bound polynucleotide 602 may have a greater number of complementary bases to the fluorophore strand 608 than the quencher strand 610 has to the fluorophore strand 608.

The device 600 may also include a fluid chamber 614 that contains the fluid 604 and keeps the fluid in contact with the substrate 108. The fluid chamber 614 may be implemented as any type of structure or material that can contain fluid. For example, the fluid chamber 614 may be implemented at a piece of tape or film that is adhered to the surface of the substrate 108 trapping the fluid 604 in between. In some implementations, the fluid chamber 614 is not a solid structure but may use hydrophobicity to create a “container” for the fluid 604 such as by creating a water-in-oil emulsion where the water portion contains the fluid 604. At least a portion of the fluid chamber 614 is clear or translucent so that fluorescence of the fluorophore 302 can be observed. In one implementation, the fluid chamber 614 may be made out of clear plastic.

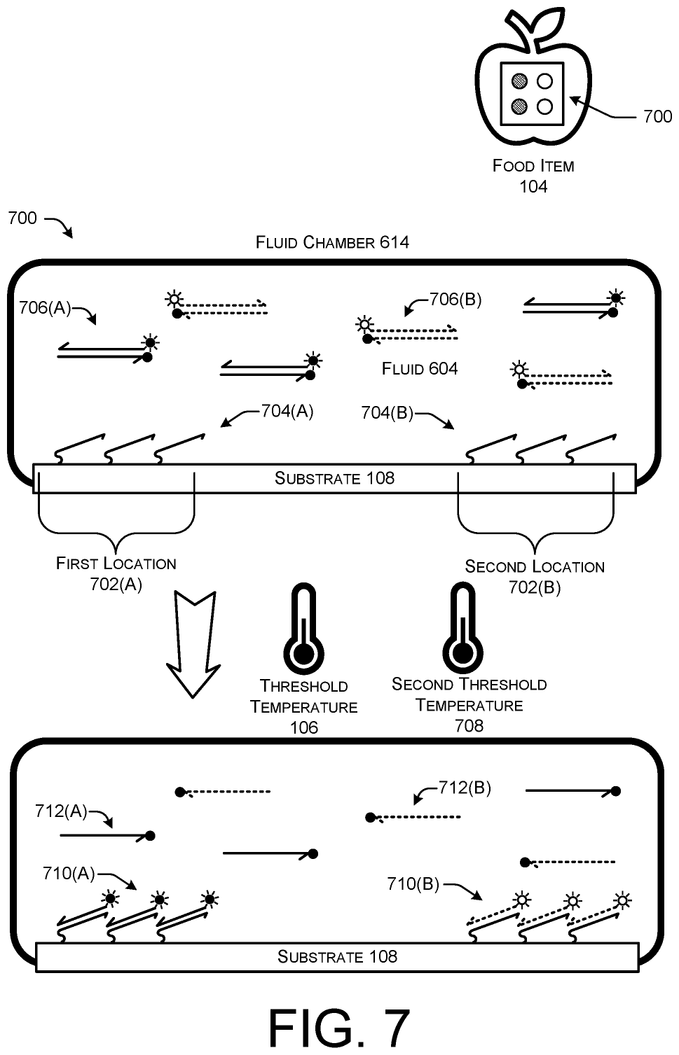

FIG. 7 illustrates an implementation of a device 700 similar to the device 600 shown in FIG. 6 but with multiple locations for fluorescence to indicate different threshold temperatures. These are illustrated as a first location 702(A) and a second location 702(B). These may correspond to the first location 202(A) and the second location 202(B) shown in FIG. 2. Although only two locations 702 are illustrated, the device 700 may have a greater number. This device 600 also includes a substrate 108, fluid 604 in contact with the substrate 108, and may have a fluid chamber 614.

Thus, on the substrate 108 there is a first location 702(A) where first single-stranded substrate-bound polynucleotides 704(A) are attached. There is also a second location 702(B), different than the first location 702(A), where second single-stranded substrate-bound polynucleotides 704(B) are attached. The single-stranded substrate-bound polynucleotides 704(A) and the second single-stranded substrate-bound polynucleotides 704(B) have different sequences which will hybridize to different polynucleotides.

The fluid 604 contains multiple types of double-stranded polynucleotide complexes 706 (i.e., polynucleotide tags)—one type for each of the locations 702. A first double-stranded polynucleotide complex 706(A) has a first fluorophore strand 710(A) attached to a fluorophore and a first quencher strand 712(A) attached to a quencher. This may be the same as the double-stranded polynucleotide complex 606 shown in FIG. 6. There is also a second double-stranded polynucleotide complex 706(B) with a second fluorophore strand 710(B) attached to a fluorophore and a second quencher strand 712(B) attached to a quencher. All double-stranded polynucleotide complexes 706 may use the same type of fluorophore. The spatial separation of the locations 702 on the substrate 108 can be used to identify threshold temperatures rather than color. However, it is also possible for the double-stranded polynucleotide complexes 706 to include different fluorophores that fluoresce at different wavelengths. Thus, each of the locations shown in FIG. 2, for example, could fluoresce with a different color.

The first double-stranded polynucleotide complex 706(A) has a nucleotide sequence with a melting temperature that is a first threshold temperature 106. The second double-stranded polynucleotide complex 706(B) has a different nucleotide sequence with a higher melting temperature that is a second threshold temperature 708. For example, the first threshold temperature 106 may be 30° C. and the second threshold temperature 708 may be 40° C. FIG. 7 illustrates changes to the arrangements of polynucleotides in the device 700 when it is exposed to temperatures that exceed both the first threshold temperature 106 and the second threshold temperature 708.

Thus, if the device 700 is heated to about the first threshold temperature 106, the first fluorophore strand 710(A) will disassociate from the first quencher strand 712(A) and become free in solution. Then when the device 700 is heated above the second threshold temperature 708 the second fluorophore strand 710(B) will disassociate from the second quencher strand 712(B) and become two separate single-stranded polynucleotides free in solution. The first substrate-bound polynucleotide 704(A) has a sequence that hybridizes to the first fluorophore strand 710(A). The second substrate-bound polynucleotide 704(B) has a sequence that hybridizes to the second fluorophore strand 710(B). The first substrate-bound polynucleotide 704(A) will not hybridize to the second fluorophore strand 710(B). Similarly, the second substrate-bound polynucleotide 704(B) will not hybridize to the first fluorophore strand 710(A).

Once the device 700 cools below the threshold temperatures 106, 708, the first fluorophore strand 710(A) and the second fluorophore strand 710(B) hybridize to the respective substrate-bound polynucleotides 704(A), 704(B) creating localized fluorescence at the first location 702(A) and the second location 702(B). Detection of fluorescence at the first location 702(A) and the second location 702(B) indicates that the food item 102 has experienced a temperature at least as high as the second threshold temperature 708.

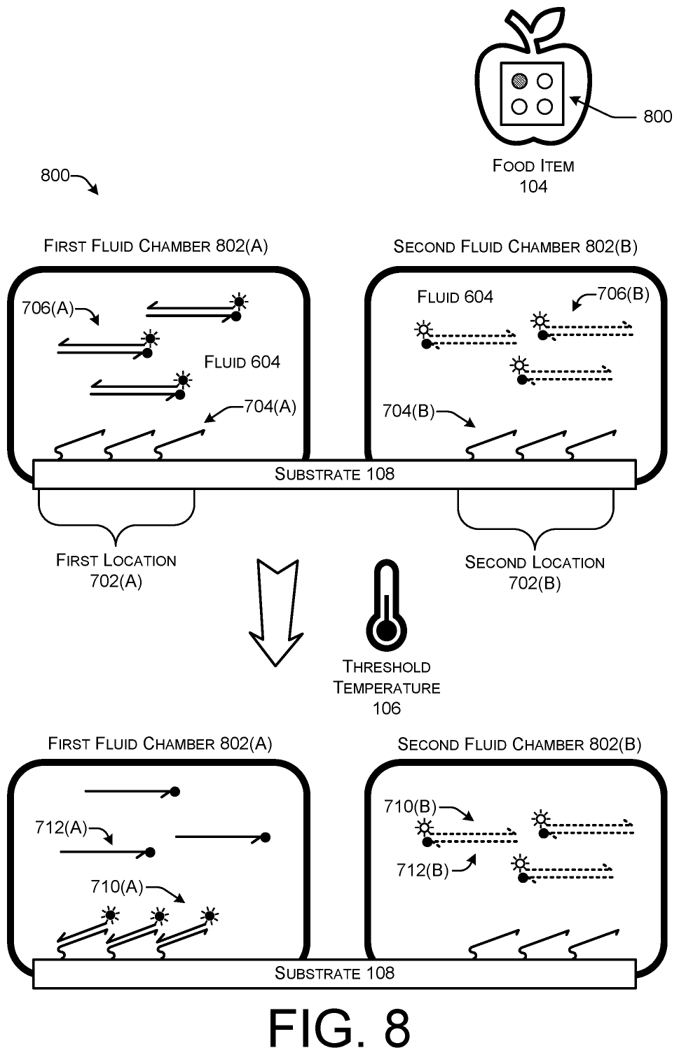

FIG. 8 illustrates an implementation of a device 800 similar to the device 700 shown in FIG. 7 but with separate fluid chambers 802 for each “spot” or location 702 on the device 800. Other components are the same. There is a first fluid chamber 802(A) covering the substrate 108 at the location of the first substrate-bound polynucleotide 704(A). The first fluid chamber 802(A) contains the fluid 604 and the first polynucleotide complex 706(A). There is also a second fluid chamber 802(B) covering the substrate 108 at the location of the second substrate-bound polynucleotide 704(B). The second fluid chamber 802(B) contains the fluid 604 and the second polynucleotide complex 706(B).

The fluid chambers are separate, that is, the first fluid chamber 802(A) and the second fluid chamber 802(B) are not in fluid communication with each other. Although the fluid 604 in both chambers will typically be identical, it is not required to be and some variation is acceptable so long as the polynucleotide tags melt at their respective threshold temperatures. Even though only two fluid chambers 802 are illustrated, there may be a greater number. Generally, there is a separate fluid chamber 802 for each spot on the substrate 108, but there may also be multiple spots with different substrate-bound polynucleotides 704 within a single fluid chamber 802 as shown in FIG. 7.

FIG. 8 also illustrates the device 800 after experiencing a temperature that exceeded the threshold temperature 106 of the first polynucleotide complex 706(A) but below the second threshold temperature of the second polynucleotide complex 706(B). Thus, the two strands of the first polynucleotide complex 706(A) have melted and at least some of the first fluorophore strands 710(A) hybridize to the first substrate-bound polynucleotides 704(A). This creates fluorescence in the first fluid chamber 802(A). However, because the second threshold temperature was not reached, the second polynucleotide complex 706(B) does not melt and there is no fluorescence in the second fluid chamber 802(B). Thus, the pattern of fluorescence on the device 800 can indicate which threshold temperatures were reached and which were not.

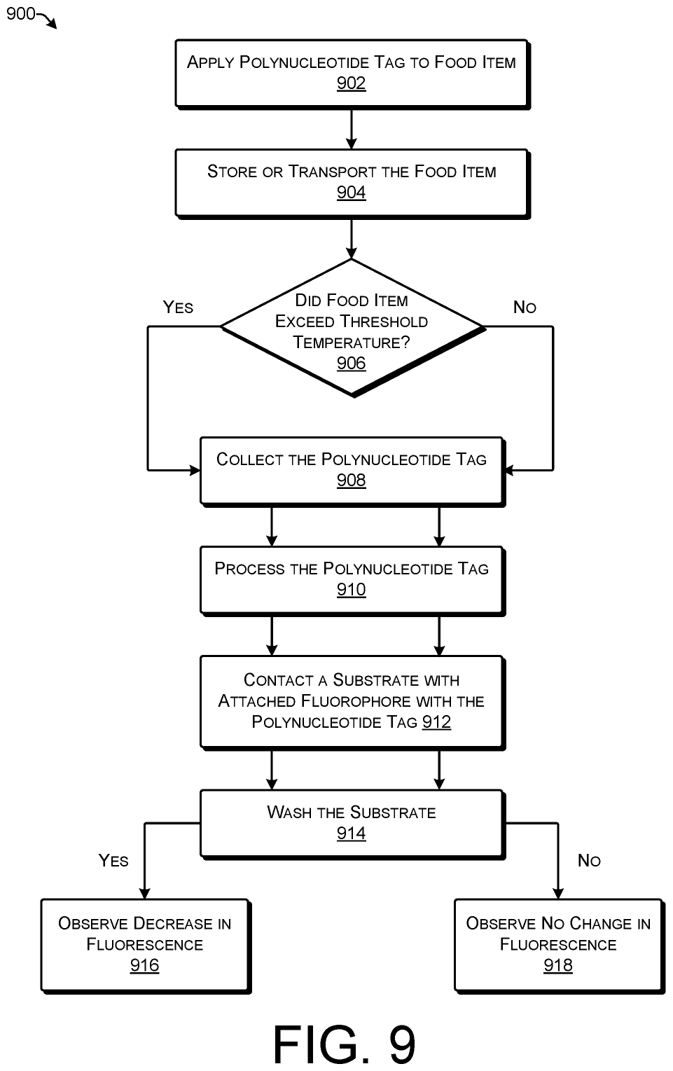

FIG. 9 is an illustrative flow diagram of an illustrative process 900 for using a polynucleotide tag to determine if a food item was exposed to temperatures that exceeded a threshold temperature.

At operation 902, an edible temperature-sensitive polynucleotide tag is applied to the food item. The temperature-sensitive polynucleotide tag may be applied to an external surface of the food item (e.g., by spraying, dipping in solution, or painting) or mixed in with the food item during manufacturing or processing. The temperature-sensitive polynucleotide tag is edible and any stabilizing chemicals may also be selected to be edible. In an implementation, the polynucleotide tag is encapsulated in the aqueous core of a microcapsule. The microcapsule may itself be edible creating an entirely edible tag.

The temperature-sensitive polynucleotide tag has a double-stranded portion that responds to exposure to the threshold temperature by melting. Also, the temperature-sensitive polynucleotide tag is configured to reanneal in a changed conformation when the temperature cools below the threshold temperature. Thus, temperature sensitivity is provided by the de-hybridization of the polynucleotide tag and a record of past temperatures is provided by the reannealing in a different conformation.

In one implementation, the polynucleotide tag comprises multiple polynucleotide strands at least one of which is partially double stranded and partially single stranded. For example, the polynucleotide tag may comprise the first polynucleotide strand 402 and the second polynucleotide strand 404 as illustrated in FIG. 4. As a further example, the polynucleotide tag may comprise the first polynucleotide strand 500 and the second polynucleotide strand 502 as illustrated in FIG. 5.

At operation 904, the food item is stored and/or transported. Generally, there is separation in both time and space between where the food item is manufactured or harvested and where it is consumed. During this time there is a risk of exposure to higher-than-ideal temperatures. The polynucleotide tag provides a record of any such exposure in a format that is closely associated with the food item.

At operation 906, if the food item experienced temperatures that exceeded the threshold temperature then process 900 proceeds along the “yes” path. If, however, the temperature experienced by the food item did not exceed the threshold temperature, then process 900 proceeds along the “no” path. The threshold temperature may be any arbitrary temperature for which reporting is desired. It may be a temperature above with the food item is damaged or quality could decrease. It could also be a temperature about which the food item would no longer be safe to consume.

Heating to or above the threshold temperature causes a conformational change in the polynucleotide tag. In the implementation illustrated in FIG. 4, a toehold blocker strand 400 disassociates from a first polynucleotide strand 402 and upon cooling below the threshold temperature, anneals to a second toehold portion 412 of a second polynucleotide strand 404. In the implementation illustrated in FIG. 5, upon heating above the threshold temperature a first blocker strand 506 disassociates a first polynucleotide strand 500, a second blocker strand 516 disassociates from a second polynucleotide strand 502. Upon cooling below the threshold temperature a first hybridized portion 510 of the first polynucleotide strand 500 hybridizes to a second hybridized portion 514 of the second polynucleotide strand 502 forming an end-hybridized complex 504.

At operation 908, the polynucleotide tags are collected from the food item. If the food item was exposed to the threshold temperature, then at least some of the polynucleotide tags will be in the changed conformation. If the food item stayed at temperatures below the threshold temperature, then the polynucleotide tags remain in their original conformation which is not the changed conformation. The polynucleotide tags may be collected using any established techniques for collecting polynucleotides such as those used to collect DNA or RNA from environmental and forensic samples. Following collection, the polynucleotide tags may be cleaned or processed using commercial kits or any one of a number of techniques known to those of ordinary skill in the art. For example, the processing may remove stabilizing agents and components of microcapsules from the polynucleotides.

At operation 910, the polynucleotide tag is processed. Not all implementations require processing. In some implementations, the change in conformation of the tag that results from heating and cooling is sufficient to cause a change in fluorescence. However, in other implementations processing may include, but is not limited to, techniques such as low-temperature polymerase extension and asymmetric PCR. The processing can create a single-stranded polynucleotide strand that is not present originally in the polynucleotide tag. This single-stranded polynucleotide strand functions as an invading strand that displaces a fluorophore.

At operation 912, a double-stranded polynucleotide structure that is attached to a substrate (e.g., a nitrocellulose membrane) and contains a fluorophore is contacted with the polynucleotide tag collected from the food item. For example, a solution containing the polynucleotide tag may be applied to a paper or other substance that is the substrate. In some implementations, the substrate may be immersed in a solution that contains the polynucleotide tag. The substrate may be incubated in contact with the polynucleotide tag to allow the polynucleotide tag time to interact with the double-stranded polynucleotide structure. The polynucleotide tag collected from the food item displaces the fluorophore. The polynucleotide tag can displace the fluorophore because of the changed conformation following heating to the threshold temperature and any processing performed at operation 910.

The double-stranded polynucleotide structure may have, for example, the structure double-stranded polynucleotide complex 300 shown in FIG. 3. For example, the double-stranded polynucleotide structure attached to the substrate may comprise a first polynucleotide strand attached to the substrate and a second polynucleotide strand hybridized to the first polynucleotide strand and attached to a fluorophore. The second polynucleotide strand has an overhang region that does not hybridize to the first polynucleotide strand. With this conformation, a single-stranded polynucleotide strand from the polynucleotide tag hybridizes along substantially its entire length to one strand of the double-stranded polynucleotide structure attached to the substrate and displaces the fluorophore. Other polynucleotide strands that may also be present in the polynucleotide tag, do not hybridize along their entire length to a strand of the double-stranded polynucleotide structure attached to the substrate and thus do not displace the fluorophore.

At operation 914, the substrate is washed to remove any displaced fluorophores. If the food item was not heated to the threshold temperature, the fluorophores will not be displaced and the fluorescence will not change. Washing can remove any polynucleotides in solution that are not attached to the substrate or hybridized to polynucleotides attached to the substrate. The washing may be performed with any fluid or solution suitable or manipulating polynucleotides such as a buffer solution. The wash solution may be the same or similar to fluids or solutions described elsewhere. To prevent de-hybridization of polynucleotides hybridized to the polynucleotides attached to the substrate, the wash solution may be cooled below the melting temperature (e.g., to 15° C., 10° C., 5° C., or 0° C.).

At operation 916, if the food item experienced temperatures that exceeded the threshold temperature, process 900 follows the “yes” path from operation 914. Washing removes the fluorophores and a decrease or loss of fluorescence is observed at a location on the substrate associated with the threshold temperature. For example, this may correspond to one of the locations 202 in FIG. 2 losing visible fluorescence.

At operation 918, alternatively if food item stayed at temperatures below the threshold temperature, process 900 follows the “no” path from operation 914. Because there are no polynucleotide strands capable of displacing the fluorophore strand, the fluorophores stay attached to the substrate and no change in fluorescence is observed.

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

The following clauses describe a method for detecting high temperature using DNA applied to a food item. The method uses processing to read the tag. Clause 1 covers both the displacement of toehold blocker to “helper” strand implementation and the low-temp polymerase followed by PCR implementation.

Clause 1. A method of detecting temperature exposure of a food item (104) comprising: (a) applying an edible temperature-sensitive polynucleotide tag (102) to the food item, wherein following exposure to a threshold temperature (106) a double-stranded portion of the polynucleotide tag melts and reanneals in a changed conformation (116); (b) after storage or transport of the food item at a temperature that exceeds the threshold temperature, collecting the polynucleotide tag in the changed conformation from the food item; and (c) contacting a double-stranded polynucleotide complex (300) attached to a substrate (108) and containing a fluorophore (302) with the polynucleotide tag collected from the food item, wherein the polynucleotide tag collected from the food item displaces the fluorophore.

Clause 2. The method of clause 1, further comprising: washing the substrate to remove the displaced fluorophore; and observing decreased fluorescence at a location on the substrate associated with the threshold temperature.

Clause 3. The method of clause 1 or 2, wherein the polynucleotide tag is encapsulated in an aqueous core of an edible microcapsule.

Clause 4. The method of any of clauses 1 to 3, wherein the polynucleotide tag comprises multiple polynucleotide strands at least one of which is partially double stranded and partially single stranded. Clause 3 provides a description of a DNA tag that applies to both the toehold blocker and PCR implementation.

Clause 5. The method of any of clauses 1 to 4, wherein the double-stranded polynucleotide complex attached to the substrate comprises: a first polynucleotide strand attached to the substrate; and a second polynucleotide strand hybridized to the first polynucleotide strand and attached to a fluorophore, wherein the second polynucleotide strand has an overhang region that does not hybridize to the first polynucleotide strand.

Clause 6. This clause provides a description of a polynucleotide tag for an implementation with a toehold blocker. The method of any of clauses 1 to 5, wherein the polynucleotide tag (102) comprises: a first polynucleotide strand (402) with a first toehold portion (408) and a first single-stranded portion (406), wherein a toehold blocker strand (400) is hybridized to the first toehold portion forming a double-stranded complex; a second polynucleotide strand (404) with a second toehold portion (412) and second single-stranded portion (410) different from the first single-stranded portion, wherein the toehold blocker strand is not hybridized to the second toehold portion; wherein a melting temperature of the first toehold portion and the toehold blocker strand is about the threshold temperature (106); and wherein the first polynucleotide strand hybridizes along substantially its entire length to one strand of the double-stranded polynucleotide complex (300) attached to the substrate (108) thereby displacing the fluorophore (302) and the second polynucleotide strand does not hybridize along its entire length to the one strand of the double-stranded polynucleotide complex attached to the substrate and does not displace the fluorophore.

Clause 7. This clause provides a description of a method and polynucleotide tags for an implementation that uses PCR. The method of any of clauses 1 to 5, further comprising after step (b) and before step (c): processing the polynucleotide tag in the changed conformation by low-temperature polymerase extension followed by asymmetric polymerase chain reaction (PCR) to create a single-stranded polynucleotide strand that is not present in the polynucleotide tag and that displaces the fluorophore.

Clause 8. The method of clause 7, wherein the polynucleotide tag comprises: a first polynucleotide strand (500) with a forward primer portion (508) and a first hybridized portion (510), wherein the first hybridized portion is hybridized to a first blocker strand (506); and a second polynucleotide strand (502) with a reverse primer portion (512) and a second hybridized portion (514), wherein the second hybridized portion is hybridized to a second blocker strand (516); wherein a melting temperature of the first polynucleotide strand and the first blocker strand is about the same as a melting temperature of the second polynucleotide strand and the second blocker strand and both are about the threshold temperature (106); wherein the first hybridized portion of the first polynucleotide strand is configured to hybridize to the second hybridized portion of the second polynucleotide strand and the first blocker strand is configured to hybridize to the second blocker strand; and wherein upon heating above the threshold temperature the first blocker strand disassociates from first polynucleotide strand, the second blocker strand disassociates from the second polynucleotide strand, and upon cooling below the threshold temperature the first hybridized portion of the first polynucleotide strand hybridizes to the second hybridized portion of the second polynucleotide strand.

The following clauses describe a paper with spots. DNA collected from the food item is applied to and will fluoresce at spots if the target temperature was exceeded.

Clause 9. A device (200) that exhibits localized changes in fluorescence when contacted by polynucleotide strands having specific melting temperatures, the device comprising: a substrate (108); a first location (202(A)) on the substrate associated with a first threshold temperature, the first location comprising a first polynucleotide strand (304) attached to the substrate and a second polynucleotide strand (306) hybridized to the first polynucleotide strand and attached to a fluorophore (302), wherein the second polynucleotide strand includes a first overhang region (310) that does not hybridize to the first polynucleotide strand; and a second location (202(B)) on the substrate associated with a second threshold temperature, the second location comprising a third polynucleotide strand (could be identical to the first) attached to the substrate and a fourth polynucleotide strand (will have to be different from the second at least at the overhang region) hybridized to the third polynucleotide strand and attached to a fluorophore, wherein the fourth polynucleotide strand includes a second overhang region that does not hybridize to the third polynucleotide strand.

Clause 10. The device of clause 9, further comprising a third location (202(C)) on the substrate associated with a third threshold temperature, the third location comprising a fifth polynucleotide strand attached to the substrate and a sixth polynucleotide strand hybridized to the first polynucleotide strand and attached to a fluorophore, wherein the third polynucleotide strand includes a third overhang region that does not hybridize to the fifth polynucleotide strand.

Clause 11. The device of clause 9 or 10, wherein the substrate comprises a nitrocellulose membrane.

Clause 12. The device of clause 11, wherein the first polynucleotide strand and the third polynucleotide strand both comprise a poly-T tail that binds to the nitrocellulose membrane.

Clause 13. The device of any of clauses 9 to 12, wherein the substrate comprises a first printed indication of the first threshold (204(A)) temperature proximate to the first location and a second printed indication of the second threshold (204(B))) temperature proximate to the second location.

Clause 14. The device of any of clauses 9 to 13, wherein: the second polynucleotide strand is configured to disassociate from the first polynucleotide strand and release the fluorophore from attachment to the substrate at the first location upon contact with a first invading strand that hybridizes to the first overhang region and to the remainder of the second polynucleotide strand; and the fourth polynucleotide strand is configured to disassociate from the third polynucleotide strand and release the fluorophore from attachment to the substrate at the second location upon contact with a second invading strand that hybridizes to the second overhang region and to the remainder of the fourth polynucleotide strand.

The following clauses describe a self-contained sensor implementation where the DNA tags are kept in contact with the surface of the paper, like a sticker or a label.

Clause 15. A device (600, 700, 800) that uses polynucleotides to report high temperature exposure comprising: a substrate (108); a single-stranded substrate-bound polynucleotide (602) attached to the substrate; a fluid (604) in contact with the surface of the substrate and the substrate-bound polynucleotide; and within the fluid, a double-stranded polynucleotide complex (606) with a fluorophore strand (608) attached to a fluorophore (302) and a quencher strand (610) attached to a quencher (612), wherein the quencher prevents fluorescence of the fluorophore, wherein the double-stranded polynucleotide complex has a nucleotide sequence with a melting temperature that is about a threshold temperature (106), and wherein the substrate-bound polynucleotide has a nucleotide sequence that hybridizes to the fluorophore strand once disassociated from the quencher strand thereby creating localized fluorescence at a location on the substrate of the substrate-bound polynucleotide if the device is exposed to a temperature that exceeds the threshold temperature.

Clause 16. The device of clause 15, wherein the threshold temperature is between about 30° C. and about 60° C.

Clause 17. The device of clause 15 or 16, wherein the device is affixed to a food item.

Clause 18. The device of any of clauses 15 to 17, further comprising a fluid chamber (614) that contains the fluid and keeps the fluid in contact with the substrate.

Clause 19. This clause adds a second “spot” to detect a second temperature. The device of any of clauses 15 to 18, further comprising: a second single-stranded substrate-bound polynucleotide (704(B)) attached to the substrate at a second location (702(B)) different than a location (702(A)) of the substrate-bound polynucleotide; and within the fluid, a second double-stranded polynucleotide complex (706(B)) with a second fluorophore strand (710(B)) attached to a fluorophore and a second quencher strand (712(B)) attached to a quencher, wherein the second double-stranded polynucleotide complex has a nucleotide sequence with a melting temperature that is about a second threshold temperature (708), wherein the second substrate-bound polynucleotide has a nucleotide sequence that hybridizes to the second fluorophore strand once disassociated from the second quencher strand thereby creating localized fluorescence at the second location on the substrate of the second substrate-bound polynucleotide if the device is exposed to a temperature that exceeds the second threshold temperature.

Clause 20. The device of clause 19, wherein the substrate comprises a printed indication of the threshold temperature proximate to the location of the substrate-bound polynucleotide and a printed indication of the second threshold temperature proximate to the second location of the second substrate-bound polynucleotide.

Clause 21. This clause describes an implementation with the liquid above each spot in separate “bubbles” rather than a single large pool. The device (800) of any of clauses 15 to 20, wherein the device further comprises: a first fluid chamber (802(A)) covering the substrate at the location of the substrate-bound polynucleotide that contains the fluid and the polynucleotide complex; and a second fluid chamber (802(B)) covering the substrate at the second location of the second substrate-bound polynucleotide that contains the fluid and the second polynucleotide complex, wherein the first fluid chamber and the second fluid chamber are not in fluid communication with each other.

### CONCLUSION

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced. As used herein, “approximately” or “about” or similar referents denote a range of ±10% of the stated value.

For ease of understanding, the processes discussed in this disclosure are delineated as separate operations represented as independent blocks. However, these separately delineated operations should not be construed as necessarily order-dependent in their performance. The order in which the processes are described is not intended to be construed as a limitation, and unless other otherwise contradicted by context any number of the described process blocks may be combined in any order to implement the process or an alternate process. Moreover, it is also possible that one or more of the provided operations is modified or omitted.

Certain embodiments are described herein, including the best mode known to the inventors for carrying out the invention. Of course, variations on these described embodiments will become apparent to those of ordinary skill in the art upon reading the foregoing description. Skilled artisans will know how to employ such variations as appropriate, and the embodiments disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

Furthermore, references have been made to publications, patents and/or patent applications throughout this specification. Each of the cited references is individually incorporated herein by reference for its particular cited teachings as well as for all that it discloses.

1. A method of detecting temperature exposure of a food item comprising:
(a) applying an edible temperature-sensitive polynucleotide tag to the food item, wherein following exposure to a threshold temperature a double-stranded portion of the polynucleotide tag melts and reanneals in a changed conformation;
(b) after storage or transport of the food item at a temperature that exceeds the threshold temperature, collecting the polynucleotide tag in the changed conformation from the food item; and
(c) contacting a double-stranded polynucleotide complex attached to a substrate and containing a fluorophore with the polynucleotide tag collected from the food item, wherein the polynucleotide tag collected from the food item displaces the fluorophore.

2. The method of claim 1, further comprising:
washing the substrate to remove the displaced fluorophore; and
observing decreased fluorescence at a location on the substrate associated with the threshold temperature.

3. The method of claim 1, wherein the polynucleotide tag is encapsulated in an aqueous core of an edible microcapsule.

4. The method of claim 1, wherein the polynucleotide tag comprises multiple polynucleotide strands at least one of which is partially double stranded and partially single stranded.

5. The method of claim 1, wherein the double-stranded polynucleotide complex attached to the substrate comprises:
a first polynucleotide strand attached to the substrate; and
a second polynucleotide strand hybridized to the first polynucleotide strand and attached to a fluorophore, wherein the second polynucleotide strand has an overhang region that does not hybridize to the first polynucleotide strand.

6. The method of claim 1, wherein the polynucleotide tag comprises:
a first polynucleotide strand with a first toehold portion and a first single-stranded portion, wherein a toehold blocker strand is hybridized to the first toehold portion forming a double-stranded complex;
a second polynucleotide strand with a second toehold portion and second single-stranded portion different from the first single-stranded portion, wherein the toehold blocker strand is not hybridized to the second toehold portion;
wherein a melting temperature of the first toehold portion and the toehold blocker strand is about the threshold temperature; and
wherein the first polynucleotide strand hybridizes along substantially its entire length to one strand of the double-stranded polynucleotide complex attached to the substrate thereby displacing the fluorophore and the second polynucleotide strand does not hybridize along its entire length to the one strand of the double-stranded polynucleotide complex attached to the substrate and does not displace the fluorophore.

7. The method of claim 1, further comprising after step (b) and before step (c):
processing the polynucleotide tag in the changed conformation by low-temperature polymerase extension followed by asymmetric polymerase chain reaction (PCR) to create a single-stranded polynucleotide strand that is not present in the polynucleotide tag and that displaces the fluorophore.

8. The method of claim 7, wherein the polynucleotide tag comprises:
a first polynucleotide strand with a forward primer portion and a first hybridized portion, wherein the first hybridized portion is hybridized to a first blocker strand; and
a second polynucleotide strand with a reverse primer portion and a second hybridized portion, wherein the second hybridized portion is hybridized to a second blocker strand;
wherein a melting temperature of the first polynucleotide strand and the first blocker strand is about the same as a melting temperature of the second polynucleotide strand and the second blocker strand and both are about the threshold temperature;
wherein the first hybridized portion of the first polynucleotide strand is configured to hybridize to the second hybridized portion of the second polynucleotide strand and the first blocker strand is configured to hybridize to the second blocker strand; and
wherein upon heating above the threshold temperature the first blocker strand disassociates from first polynucleotide strand, the second blocker strand disassociates from the second polynucleotide strand, and upon cooling below the threshold temperature the first hybridized portion of the first polynucleotide strand hybridizes to the second hybridized portion of the second polynucleotide strand.

9. A device that exhibits localized changes in fluorescence when contacted by polynucleotide strands having specific melting temperatures, the device comprising:
a substrate;
a first location) on the substrate associated with a first threshold temperature, the first location comprising a first polynucleotide strand attached to the substrate and a second polynucleotide strand hybridized to the first polynucleotide strand and attached to a fluorophore, wherein the second polynucleotide strand includes a first overhang region that does not hybridize to the first polynucleotide strand; and
a second location) on the substrate associated with a second threshold temperature, the second location comprising a third polynucleotide strand (could be identical to the first) attached to the substrate and a fourth polynucleotide strand (will have to be different from the second at least at the overhang region) hybridized to the third polynucleotide strand and attached to a fluorophore, wherein the fourth polynucleotide strand includes a second overhang region that does not hybridize to the third polynucleotide strand.

10. The device of claim 9, further comprising a third location) on the substrate associated with a third threshold temperature, the third location comprising a fifth polynucleotide strand attached to the substrate and a sixth polynucleotide strand hybridized to the first polynucleotide strand and attached to a fluorophore, wherein the third polynucleotide strand includes a third overhang region that does not hybridize to the fifth polynucleotide strand.

11. The device of claim 9, wherein the substrate comprises a nitrocellulose membrane.

12. The device of claim 9, wherein the substrate comprises a first printed indication of the first threshold) temperature proximate to the first location and a second printed indication of the second threshold)) temperature proximate to the second location.

13. The device of claim 9, wherein:
the second polynucleotide strand is configured to disassociate from the first polynucleotide strand and release the fluorophore from attachment to the substrate at the first location upon contact with a first invading strand that hybridizes to the first overhang region and to the remainder of the second polynucleotide strand; and
the fourth polynucleotide strand is configured to disassociate from the third polynucleotide strand and release the fluorophore from attachment to the substrate at the second location upon contact with a second invading strand that hybridizes to the second overhang region and to the remainder of the fourth polynucleotide strand.

14. A device that uses polynucleotides to report high temperature exposure comprising:
a substrate;
a single-stranded substrate-bound polynucleotide attached to the substrate;
a fluid in contact with the surface of the substrate and the substrate-bound polynucleotide; and
within the fluid, a double-stranded polynucleotide complex with a fluorophore strand attached to a fluorophore and a quencher strand attached to a quencher, wherein the quencher prevents fluorescence of the fluorophore,
wherein the double-stranded polynucleotide complex has a nucleotide sequence with a melting temperature that is about a threshold temperature, and
wherein the substrate-bound polynucleotide has a nucleotide sequence that hybridizes to the fluorophore strand once disassociated from the quencher strand thereby creating localized fluorescence at a location on the substrate of the substrate-bound polynucleotide if the device is exposed to a temperature that exceeds the threshold temperature.

15. The device of claim 14, wherein the threshold temperature is between about 30° C. and about 60° C.

16. The device of claim 14, wherein the device is affixed to a food item.

17. The device of claim 14, further comprising a fluid chamber that contains the fluid and keeps the fluid in contact with the substrate.

18. The device of claim 14, further comprising:
a second single-stranded substrate-bound polynucleotide) attached to the substrate at a second location) different than a location) of the substrate-bound polynucleotide; and
within the fluid, a second double-stranded polynucleotide complex) with a second fluorophore strand) attached to a fluorophore and a second quencher strand) attached to a quencher,
wherein the second double-stranded polynucleotide complex has a nucleotide sequence with a melting temperature that is about a second threshold temperature,
wherein the second substrate-bound polynucleotide has a nucleotide sequence that hybridizes to the second fluorophore strand once disassociated from the second quencher strand thereby creating localized fluorescence at the second location on the substrate of the second substrate-bound polynucleotide if the device is exposed to a temperature that exceeds the second threshold temperature.

19. The device of claim 18, wherein the substrate comprises a printed indication of the threshold temperature proximate to the location of the substrate-bound polynucleotide and a printed indication of the second threshold temperature proximate to the second location of the second substrate-bound polynucleotide.

20. The device of claim 18, wherein the device further comprises:
a first fluid chamber) covering the substrate at the location of the substrate-bound polynucleotide that contains the fluid and the polynucleotide complex; and
a second fluid chamber) covering the substrate at the second location of the second substrate-bound polynucleotide that contains the fluid and the second polynucleotide complex, wherein the first fluid chamber and the second fluid chamber are not in fluid communication with each other.
